(19) United States
(12) Patent Application Publication
(10) Pub. No.: US 2010/0126041 A1
(43) Pub. Date: May 27, 2010

(54) Foot-Bed

(75) Inventor: Stephen A. Francis, Newburyport, MA (US)

Correspondence Address: JOHN S. PRATT, ESQ;KILPATRICK STOCKTON, LLP, 1100 PEACHTREE STREET, SUITE 2800, ATLANTA, GA, 30309, US

(73) Assignee: Altama Footwear, Atlanta, GA (US)

(21) Appl. No.: 12/620,256

(22) Filed: Nov. 17, 2009

Related U.S. Application Data
(60) Provisional application No. 61/115,196, filed on Nov. 17, 2008.

(51) Int. Cl. A43B 7/22; A43B 13/38; A61F 5/14; A43B 7/06

(52) U.S. Cl. 36/91; 36/44; 36/147; 36/3 B

(57) ABSTRACT

A foot-bed, also called an innersole or sock liner, having a two-piece molded innersole with an upper section, a lower section, and an optional mesh cover (or top liner). The lower surface of the upper section of the innersole has a specific contour that follows the bottom surface of the foot. The upper section of the innersole fits into a cavity in the lower section of the innersole. The lower section is cupped in the cross-section and provides support and stability.

### CROSS REFERENCE TO RELATED APPLICATIONS

This application claims priority to and incorporates by reference U.S. provisional application No. 61/115,196, filed Nov. 17, 2008.

### FIELD OF THE INVENTION

This invention relates generally to footwear. More specifically, the present invention relates to a foot-bed, also called an innersole or sock liner. The present invention also relates to a footwear device comprising an item of footwear, such as a shoe or a boot, and an innersole that can be attached to the footwear device.

### BACKGROUND

Shoes are designed to protect the foot and provide support and comfort for the wearer. Shoes may need to be worn for long periods of time and must remain comfortable for the wearer. Shoes must also provide adequate support to help the individual avoid injury. It is also desirable for the shoes to help maintain the wearer's feet in a dry and comfortable state despite normal or excessive perspiration or damp environmental conditions. The resulting perspiration and warm temperatures can encourage the growth of bacteria and other organisms, leading to potential risk of infection or undesirable odors. Furthermore, for individuals in certain occupations, such as law enforcement or the military, shoes are particularly valuable for protecting the wearer's feet and are an important part of the uniform.

A foot-bed, also called an innersole or sock liner, may be incorporated into some types of footwear. A foot-bed is typically layered on top of the sole of the shoe. The foot-bed may have cushioning or support elements, and the wearer's foot may rest directly on top of the foot-bed. Consequently, it is important for the foot-bed to be comfortable, supportive, and to keep the wearer's feet dry. However, foot-beds currently found in the art are not satisfactory for all of these purposes.

For example, wearing footwear may cause problems for the wearer over time if the footwear is not sufficiently supportive or comfortable. Many occupations require a worker to be on his or her feet for long hours without the opportunity for rest. For individuals in certain occupations, such as law enforcement or the military, specific shoes may be required.

Therefore, there is a need in the art for a foot-bed which provides support and stability while also providing comfort and cushioning.

There is also a need in the art for a foot-bed which provides improved-air circulation and drainage to keep moisture away from the foot.

There is also a need for a foot-bed that provides improved ventilation to the foot, has anti-microbial properties, and that can be removed from the footwear for drainage or washing and drying to improve comfort and hygiene of the footwear.

### SUMMARY

Embodiments of this invention provide a foot-bed, also called an innersole or sock liner, designed to be used in footwear. In one embodiment, the foot-bed includes a two-piece molded innersole with an upper section, a lower section, and an optional mesh cover (or top liner). The lower surface of the upper section of the innersole has a specific contour that follows the bottom surface of the foot. The upper section of the innersole fits into a cavity in the lower section of the innersole. The lower section is cupped in the cross-section and provides support and stability.

In one embodiment, the upper section and the lower section include a plurality of through holes, which allow air circulation and drainage. Foot-beds of this invention are easily removable from footwear.

### BRIEF DESCRIPTION OF DRAWINGS

FIG. 1 is an exploded perspective view of one embodiment of a foot-bed according to this invention.

FIG. 2 is a top view of a foot-bed according to an embodiment of this invention, shown without a mesh liner.

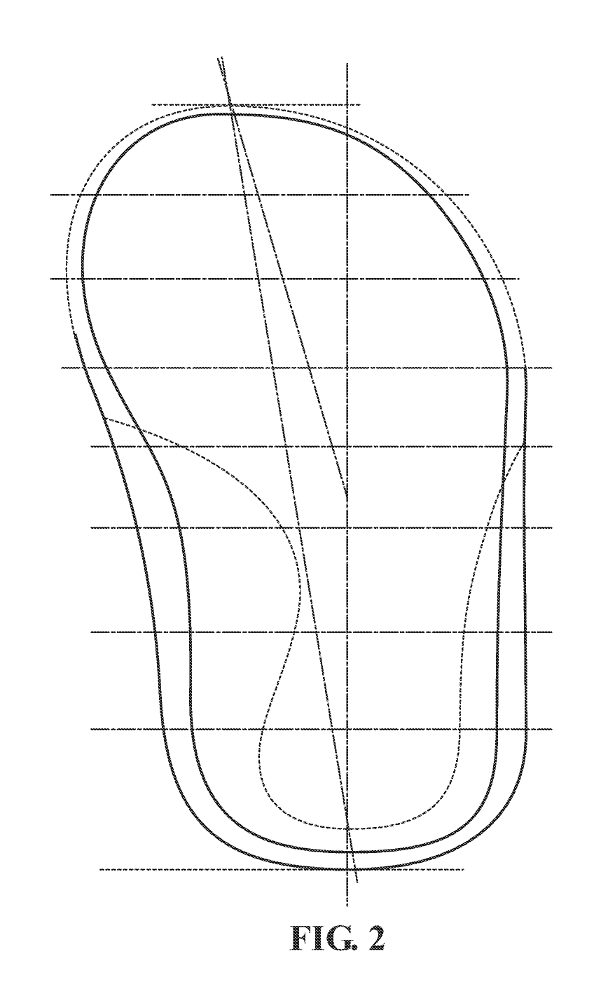

FIG. 3 is a bottom view of the foot-bed of FIG. 2.

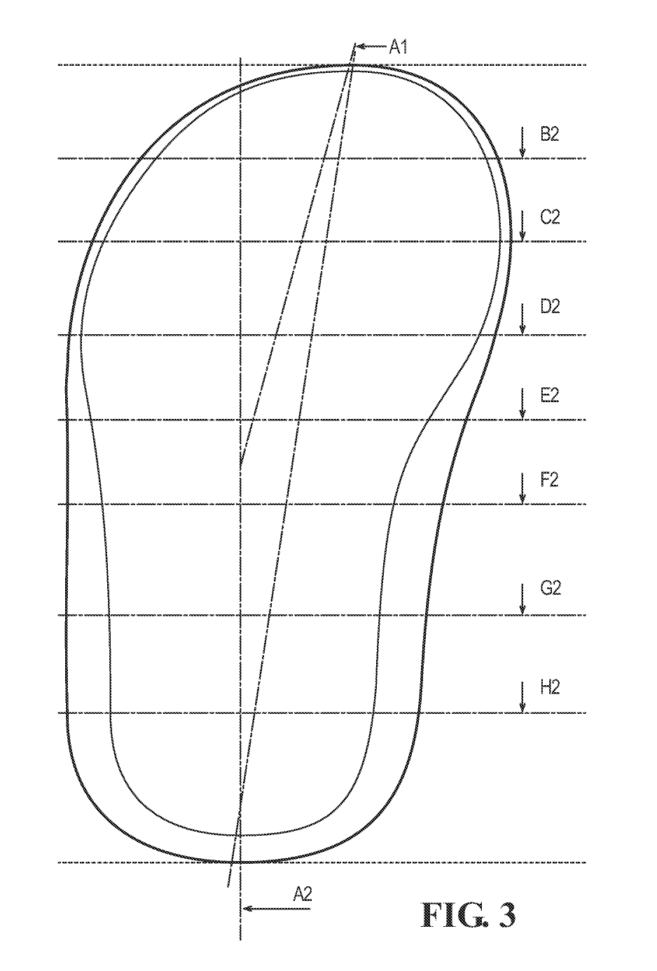

FIG. 4 is a cross-sectional end view of the foot-bed of FIG. 3, taken along line B2.

FIG. 5 is a cross-sectional end view of the foot-bed of FIG. 3, taken along line C2.

FIG. 6 is a cross-sectional end view of the foot-bed of FIG. 3, taken along line D2.

FIG. 7 is a cross-sectional end view of the foot-bed of FIG. 3, taken along line E2.

FIG. 8 is a cross-sectional end view of the foot-bed of FIG. 3, taken along line F2.

FIG. 9 is a cross-sectional end view of the foot-bed of FIG. 3, taken along line G2.

FIG. 10 is a cross-sectional end view of the foot-bed of FIG. 3, taken along line H2.

FIG. 11 is a side lateral view of the foot-bed.

FIG. 12 is a side medial view of the foot-bed.

FIG. 13 is a cross-sectional side view of the foot-bed.

FIG. 14 is a representation of the top and medial views of the foot-bed drawn to 50% scale.

### DETAILED DESCRIPTION OF THE INVENTION

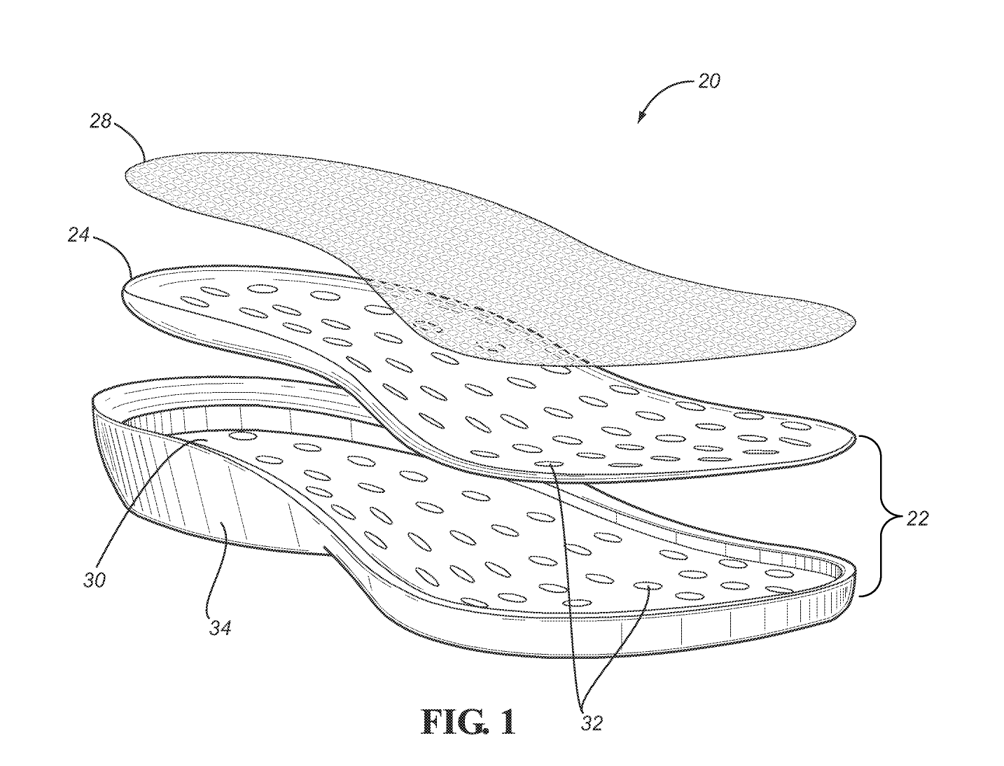

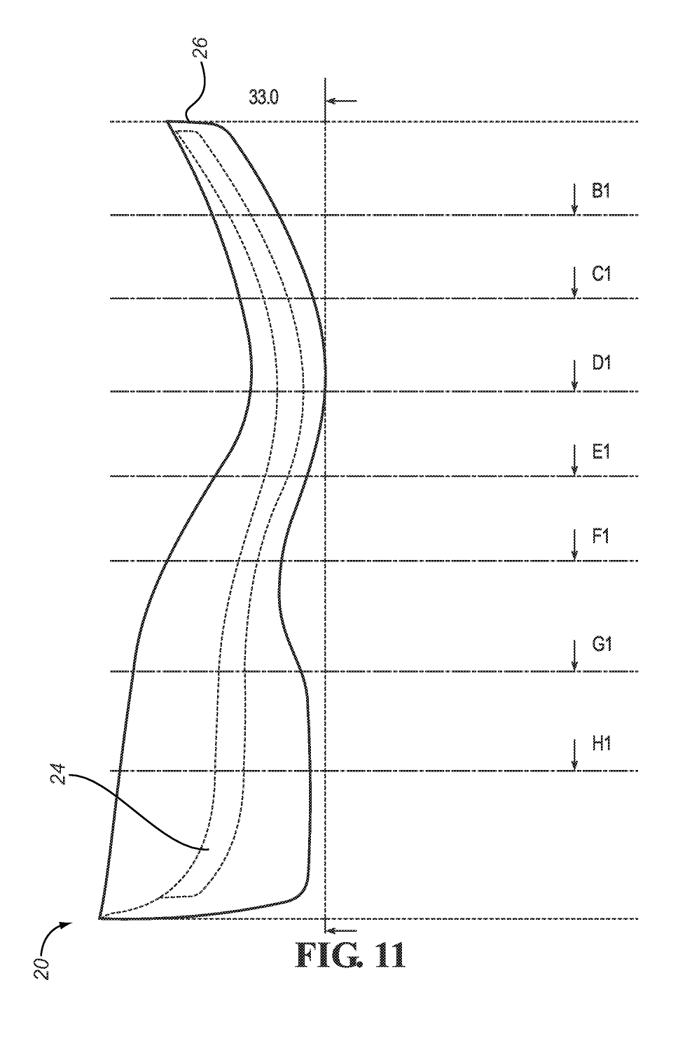

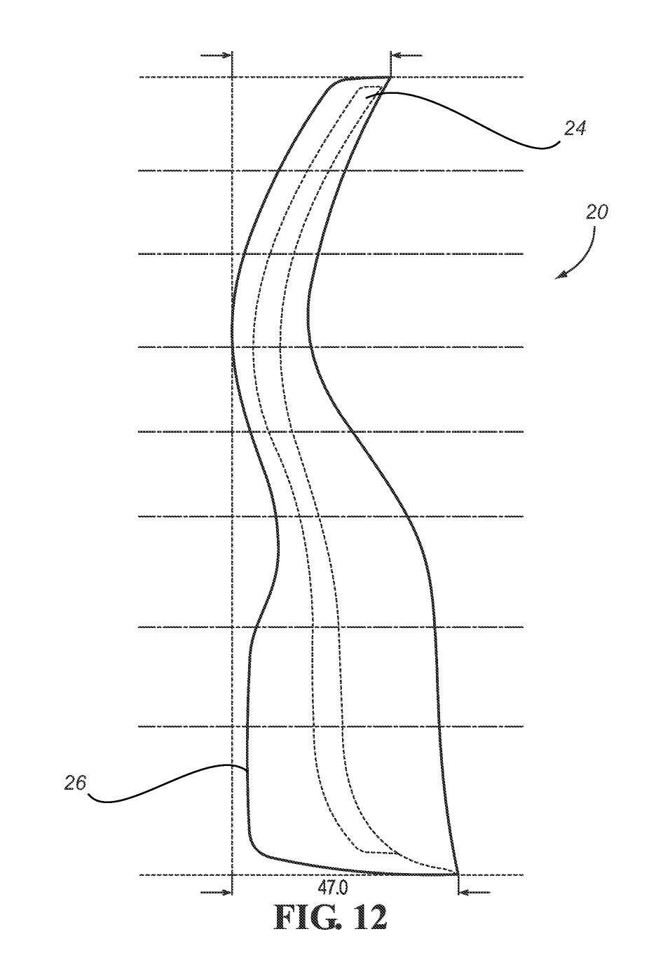

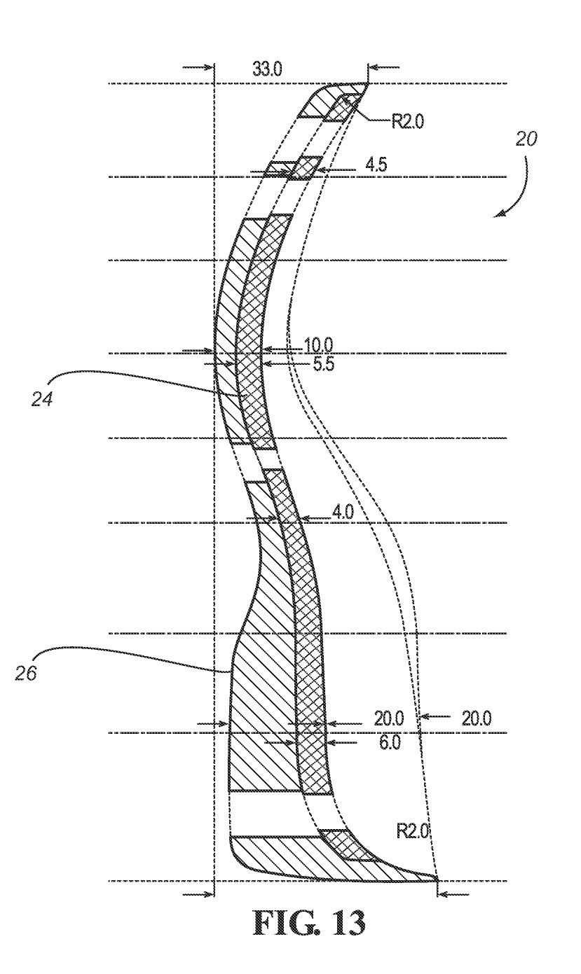

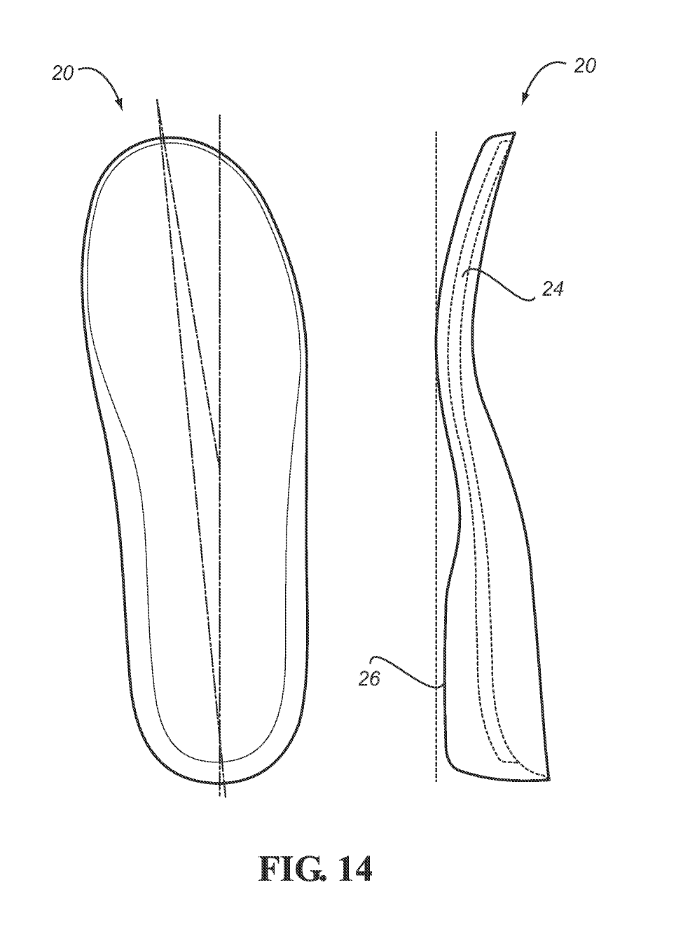

As shown in the Figures, embodiments of this invention include a foot-bed 20. As shown in FIG. 1, the foot-bed 20 includes a two-piece molded innersole 22 having an upper section 24, a lower section 26, and an optional mesh cover (or top liner) 28. FIGS. 1 and 11-14 illustrate that the lower surface 25 of the upper section 24 of the innersole 22 is contoured to mimic the contour of the bottom surface of a foot. FIG. 1 illustrates the upper section 24 of the innersole 22, which fits into a cavity 30 in the lower section 26 of the innersole 22. As shown in FIGS. 1 and 11-14 the lower section 26 has a cupped shape in cross-section, providing support and stability to a wearer.

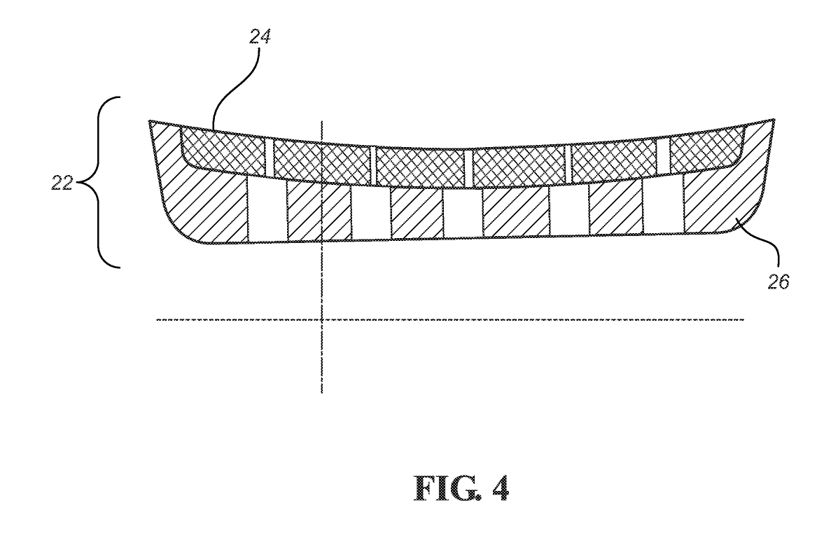

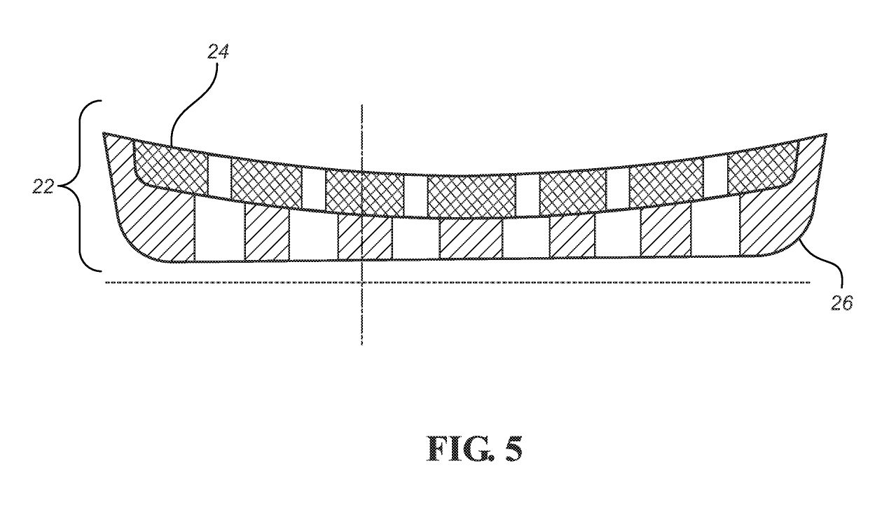

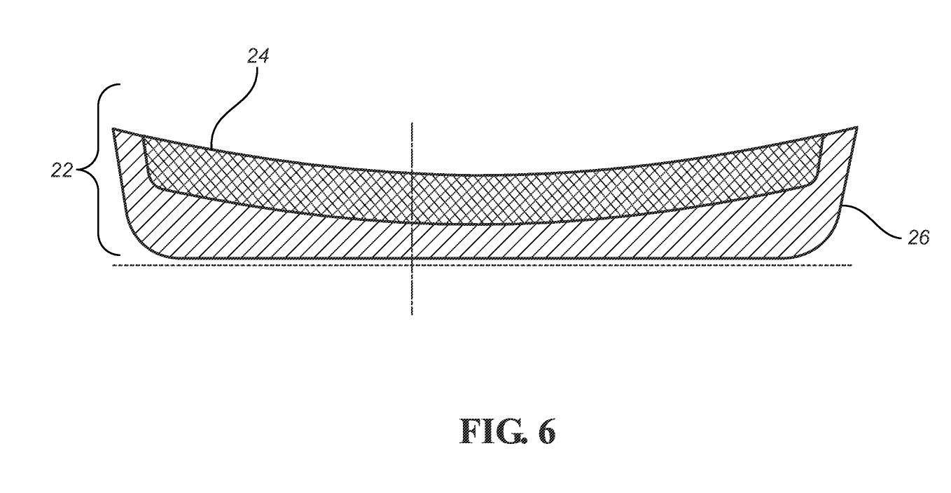

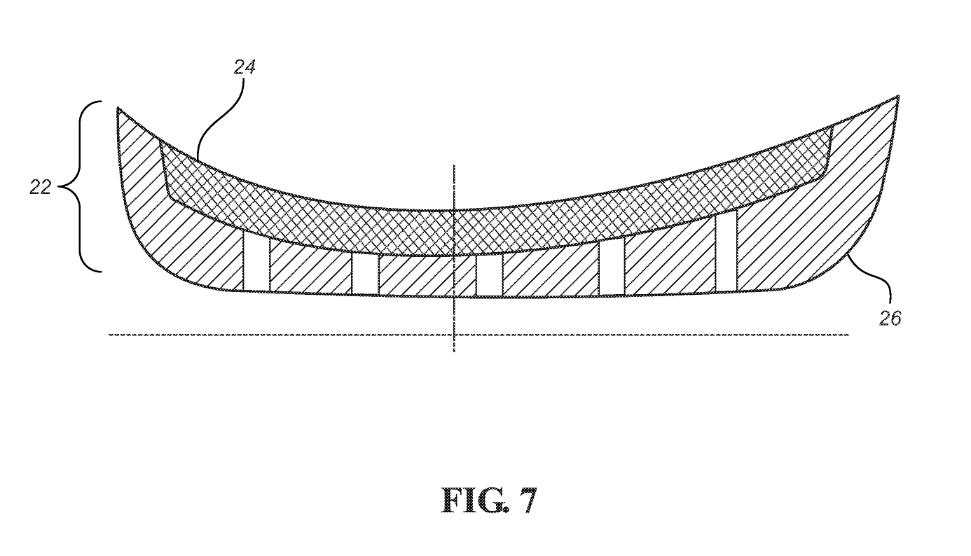

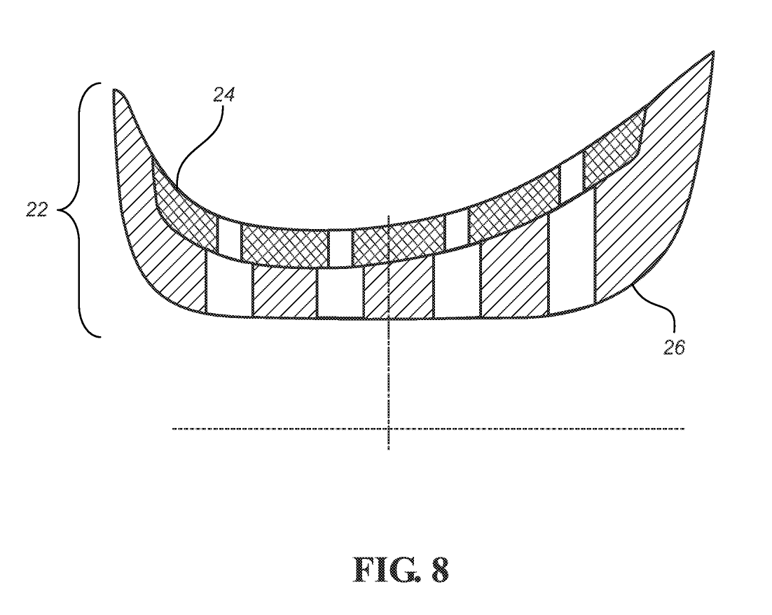

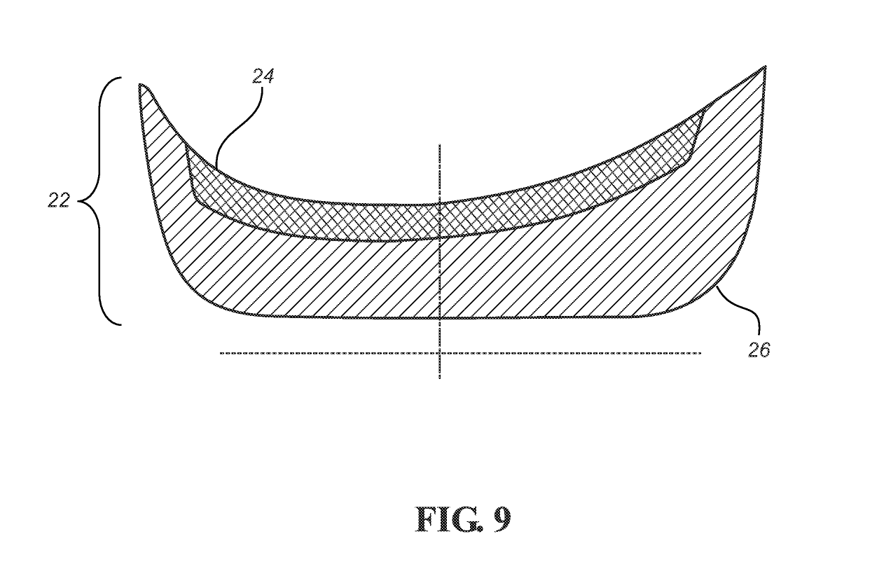

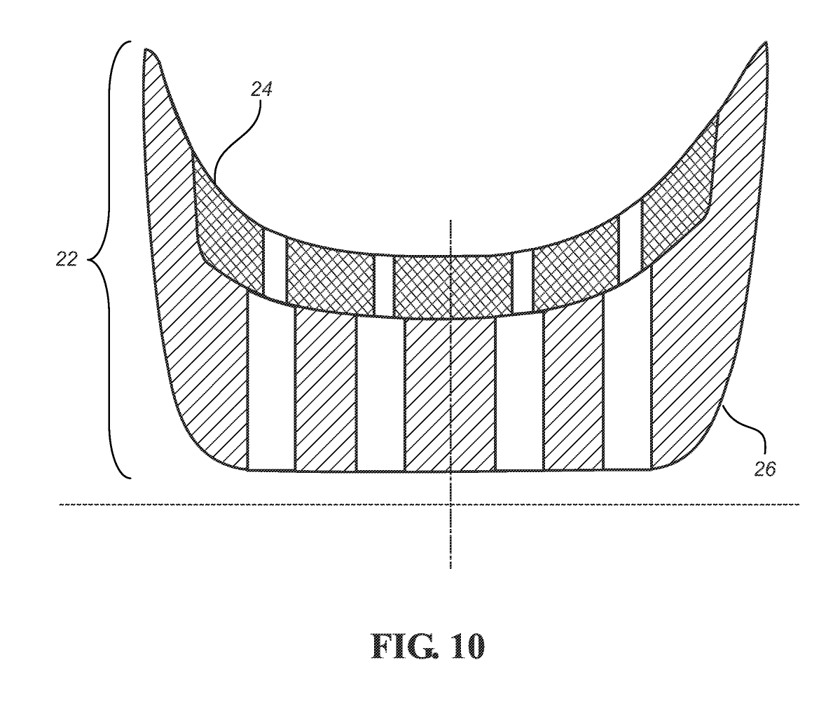

As illustrated in FIGS. 1 and 11-14, both the upper section 24 the lower section 26 are “multi-contoured,” so that the cross-sections of each section do not have a uniform thickness. In specific embodiments of the invention and as illustrated in FIGS. 1 and 4-14, the assembled foot-bed is unusually thick, with more of the bottom support material under the arch of the foot and more of the soft upper layer in the heel pocket than an average foot-bed. In one embodiment of the foot-bed this may be called an “adaptive arch.” One embodiment of the foot-bed is two inches thick at its thickest point to provide extra cushioning and support for certain parts of the foot such as the arch and heel

In another embodiment, a top liner 28 is used. The top liner 28 covers the upper section 24, as shown in FIG. 1. Top liner 28 may be formed from a woven material, or any other suitable material. In one embodiment, the top liner 28 is formed from a woven material, such as an open screen mesh, that prevents the foot from sliding, thus limiting friction and resulting moisture build-up from perspiration. The top liner 28 thus reduces friction and encourages moisture wicking away from the foot, leading to improved dryness and comfort. In some embodiments, the top liner also has anti-microbial properties. The entire foot-bed is easily removable from the shoe for drainage, drying, and washing.

As shown in FIG. 1, some embodiments of a foot-bed 20 include a plurality of holes 32 that go all the way through both the upper section 24 and the lower section 26. Alignment of the holes 32 of each section 24, 26 allows air to circulate and moisture to drain away from the foot. Thus, the holes 32 increase air circulation and improves moisture collection to the bottom of the foot-bed 20 and away from the foot (i.e., improves drainage), leading to advantageous multi-port ventilation and providing improved dryness and comfort.

In one embodiment, illustrated in FIG. 1, the upper section 24 is softer than the lower section 26 for cushioning. In one embodiment, the upper section is molded with a “cushion” of a material such as polyurethane or molded EVA foam, and the bottom support frame 34 of the lower section 26 is molded from a material such as double density EVA, blown EVA, injected EVA, or rubber, which provides support and limits compression. In one embodiment of the foot-bed, the material used for the lower section 26 is injection EVA. Both the upper 24 and lower 26 sections are “multi-contoured” such that the cross-sections are not of uniform thickness. In this embodiment of the foot-bed, the assembled foot-bed is unusually thick compared to other foot-beds and has an “adaptive arch”, with more of the bottom support material under the arch of the foot and more of the soft upper layer in the heel pocket than an average foot-bed, with a deep heel pocket for improved fit and comfort.

One embodiment of the invention may be a footwear device, formed from an item of footwear, such as a shoe or a boot, with the foot-bed or innersole attached inside. The Altama® Ortho TacX® line is an example of such a footwear device. As noted above, the entire foot-bed is easily removable from the shoe for drainage, drying, and washing. It can replace the cushioning elements that are normally permanently affixed inside a shoe or a boot.

It should be understood that the foregoing relates only to certain embodiments of the present invention and that numerous modifications or alterations may be made therein without departing from the spirit and the scope of the present invention.

1. An innersole for use with footwear, comprising:
(a) a first layer comprising a contoured top surface, a contoured bottom surface, and a plurality of holes through both surfaces, and
(b) a second layer comprising
a contoured surface having a complementary contour to the contour of the contoured bottom surface of the first layer,
a cavity that houses the first layer, and
a plurality of holes that penetrate the second layer that are in line with the plurality of holes through both surfaces of the first layer,
wherein the first layer is housed in the cavity of the second layer so that the insert provides increased cushioning and support in a heel section of the innersole.

2. The innersole of claim 1, further comprising a top liner formed from a woven material, such as mesh.

3. The innersole of claim 2, wherein the top liner has antimicrobial properties.

4. The innersole of claim 1, further comprising an arch section, wherein the arch section includes an increased amount of cushioning compared to other sections of the innersole.

5. The innersole of claim 1, wherein the innersole is removable from footwear.

6. The innersole of claim 1, wherein the first layer is formed from molded EVA foam and the second layer is formed from injected EVA or rubber.

7. A footwear device comprising:
(a) an item of footwear such as a boot or a shoe; and
(b) an innersole for use with footwear, further comprising:
(1) a first layer comprising a contoured top surface, a contoured bottom surface, and a plurality of holes through both surfaces, and
(2) a second layer comprising
a contoured surface having a complimentary contour to the contour of the contoured bottom surface of the first layer,
a cavity that houses the first layer, and
a plurality of holes that penetrate the second layer that are in line with the plurality of holes through both surfaces of the first layer,
wherein first layer is housed in the cavity of the second layer so that the insert provides increased cushioning and support in a heel section of the insert.

8. The footwear device of claim 7, wherein the innersole further comprises a top liner formed from a woven material, such as mesh.

9. The footwear device of claim 8, wherein the top liner has antimicrobial properties.

10. The footwear device of claim 7, wherein the innersole further comprises an arch section, wherein the arch section includes an increased amount of cushioning compared to other sections of the innersole.

11. The footwear device of claim 7, wherein the innersole is removable from the item of footwear.

12. The footwear device of claim 7, wherein the first layer is formed from molded EVA foam and the second layer is formed from injected EVA or rubber.

13. A footwear device comprising:
(a) an item of footwear such as a boot or a shoe; and
(b) an innersole for use with footwear, comprising:
(1) a first layer, formed from molded EVA foam, comprising a contoured top surface, a contoured bottom surface, and a plurality of holes through both surfaces, and
(2) a second layer, formed from injected EVA, comprising
a contoured surface having a complimentary contour to the contour of the contoured bottom surface of the first layer,
a cavity that houses the first layer, and
a plurality of holes that penetrate the second layer that are in line with the plurality of holes through both surfaces of the first layer,
wherein first layer is housed in the cavity of the second layer so that the insert provides increased cushioning and support in a heel section of the insert, and
(3) a top liner formed from a woven material, wherein the top liner has antimicrobial properties, and
(4) an arch section, wherein the arch section includes an increased amount of cushioning compared to other sections of the innersole;
wherein the innersole is removable from the item of footwear.
